(19) United States
(12) Patent Application Publication
(10) Pub. No.: US 2011/0218604 A1
(43) Pub. Date: Sep. 8, 2011

(54) Cardiac Lead for Epicardial, Endocardial and Trans-Coronary Sinus Placement

(76) Inventors: Sen Ji, La Canada, CA (US); Danya Zhang, La Canda, CA (US)

(21) Appl. No.: 13/037,623

(22) Filed: Mar. 1, 2011

Related U.S. Application Data
(60) Provisional application No. 61/309,475, filed on Mar. 2, 2010.

(51) Int. Cl. A61N 1/05

(52) U.S. Cl. 607/120; 607/128

(57) ABSTRACT

A cardiac lead includes a half-domed or semi-spherical shaped distal assembly and an active fixation mechanism. The half-domed or semi-spherical shape provides directionality as to whether a flat side of the lead is facing the myocardial tissue and therefore the active fixation mechanism can be deployed safely into the myocardial tissue.

This application claims priority of provisional application U.S. Ser. No. 61/309,475 filed on Mar. 2, 2010 in the U.S. Patent and Trademark Office, the entirety of which is incorporated herein by reference.

### FIELD OF THE INVENTION

The present invention relates generally to a cardiac lead. More particularly, the present invention relates to a cardiac lead having an active fixation mechanism and a half-domed or semi-spherical tip that provides lead directionality and increased precision of lead placement.

### BACKGROUND OF THE INVENTION

A normal human heart contracts rhythmically to pump blood through the circulatory system, under the control of a native cardiac pacemaking and conduction system. Abnormal function of this pacemaking and conduction system and its controlled contractile system may result in arrhythmias and dyssynchronization of the contraction. When such abnormalities occur, the treatment is often cardiac pacing, defibrillation, and/or resynchronization therapy.

To perform these treatments, cardiac rhythm management devices are implanted in patients with one or more electric leads having an active fixation mechanism. The electrical leads are placed at different locations inside (endocardial) and outside (epicardial) the heart to sense cardiac electrical activities and to deliver electrical impulses to pace, defibrillate, or resynchronize the heart.

The ventricular leads have been traditionally placed in the right ventricular apex. However, pacing at this position of the heart has been associated with a high rate of heart failure. Placing a cardiac pacing lead at the high ventricular septum may cause less dyssynchrony, and therefore less heart failure than right ventricular pacing. However, this practice is limited due to a high dislodgement rate when a conventional pacing lead is placed at the high septum. In addition, cardiac perforation is also a concern with currently available leads, as the leads extend the fixation screw straight forward into the myocardium. See U.S. Pat. No. 4,570,642; U.S. Pat. No. 5,837,006; U.S. Patent Application Publication 2006/0122682; and U.S. Patent Application Publication 2007/0129782.

U.S. Pat. No. 5,571,162 discloses a hook-like fixation mechanism in which hooks are attached to a cylindrical electrode. It is impossible to know the location of the hooks until they are exposed from the electrode. Further, if the hooks are exposed during the implant but before fixation into the myocardium, the fixation mechanism poses a high risk of lacerating the myocardium or the vessel wall if it is placed into a vein. Additionally the plurality of multiple hooks may be too stiff to be placed into a small coronary vein. All the leads disclosed in the above U.S. patents and patent publications cannot be placed to the epicardium or into a coronary vein.

Cardiac resynchronization therapy with biventricular defibrillator or pacemaker offers proven benefits to patients with severe drug-refractory heart failure. It requires that a cardiac lead be placed to the left ventricle through the coronary vein or by surgery such as thoracotomy. The technique of implanting the device has been improved, but the rate of patients responding to the therapy remains at 60 to 70%. One of the main reasons for the low response rate is that the left ventricular lead cannot be placed to the myocardial site where the most dyssynchronized, or latest, contraction is located, due to the anatomic limitation of the coronary veins. High dislodgement of coronary vein leads is another concern.

A cardiac lead may be placed to the epicardium surgically. However, surgical epicardial leads (such as disclosed in U.S. Pat. Nos. 5,143,090 and 7,270,669), require a rigid delivery sheath to fixate the leads into the myocardium. Such surgical epicardial leads therefore have the limitation of being placed only to the part of the surgically exposed myocardium, not the part of the myocardium with the latest contraction (or the most dyssynchronization).

### SUMMARY OF THE INVENTION

According to an embodiment of the present invention, a cardiac lead is provided with a distal assembly and active fixation mechanism configured for placement in the heart, particularly endocardially, epicardially and intravenously in coronary veins. The cardiac lead comprises a half-domed or semi-spherical shaped distal assembly to provide directionality to the myocardium.

In specific embodiments, disposed in the half-domed or semi-spherical distal assembly are fixation hooks, a central guide-wire channel, and a steroid delivery reservoir. The fixation hooks may be actively deployed into or retracted from the myocardium or tissue through a rotatable or pushable mechanism.

An objective of the invention is to enable a cardiac lead to be placed in the heart epicardially, endocardially and intravenously inside the coronary veins, while providing operators with the knowledge of the precise relationship of the active fixation mechanism to the heart tissue.

An advantage of the present invention is that the cardiac lead allows for placing a fixation mechanism exactly onto the septum, any site from distal to high septum, as its half-domed tip provides the directionality between the lead, its fixation mechanism, and the septum.

A further advantage of the present invention is that the fixation mechanism may be tangentially deployed and, with a distal tip coated with soft bio-compatible material such as silicon, or polyurethane, the possibility of perforation due to forward movement of the lead is avoided.

An additional advantage of the present invention is that it allows for maximally exposing the fixation mechanism to anchor in the myocardium without being hampered by adjacent cylindrical structures.

A further advantage of the present invention is that the cardiac lead may be placed at any site on the epicardium with the latest contraction, as it can be delivered by a flexible safe sheath, instead of a rigid delivery system.

Another advantage of the present invention is that the cardiac lead comprises a steroid delivery reservoir at the distal assembly to suppress scarring.

Another advantage of the present invention is that the active fixation mechanism avoids dislodgement.

Still another advantage of the present invention is that the active fixation mechanism may be deployed into the myocardium from inside a branch of the coronary vein as the lead's half-domed tip provides the directionality between the lead, its fixation mechanism and the myocardium, therefore avoiding lacerating the free wall of the vein, reinforcing the contact of the lead with the myocardium, and reducing pacing threshold. Additionally, the half-domed tip may avoid blockage of the blood flow in the vein, which may eventually cause thrombosis.

Given the following enabling description of the drawings, the apparatus and methods should become evident to a person of ordinary skill in the art.

### BRIEF DESCRIPTION OF THE DRAWINGS

FIG. 1 illustrates a perspective view of a cardiac lead as implanted to the epicardium through a sheath placed by pericardial puncture or mini-thoracotomy according to an embodiment of the present invention.

FIG. 2 illustrates a perspective view of a cardiac lead as implanted intravascularly in a cardiac coronary vein according to an embodiment of the present invention.

FIG. 3 illustrates a perspective view of a cardiac lead as implanted on the ventricular septum from the right ventricle intravascularly according to an embodiment of the present invention.

FIG. 4 illustrates a longitudinal cross-sectional view of a cardiac lead according to at least one embodiment of the present invention.

FIG. 5 illustrates a cross-sectional view of FIG. 4 showing a steroid reservoir and guide wire channel according to an embodiment of the present invention.

FIG. 6 illustrates a cross-sectional view of FIG. 4 showing an active fixation mechanism according to an embodiment of the present invention.

FIG. 7 illustrates a view of the end of the distal assembly of FIG. 4 showing an end coating of a half-domed tip of a cardiac lead.

FIG. 8 illustrates a longitudinal cross-sectional view of a cardiac lead with deployed rotatably attachable fixation hooks according to an embodiment of the present invention. It further illustrates a cardiac defibrillator lead may be incorporated.

FIG. 9 illustrates a cross-sectional view of FIG. 8 showing the rotatable attachable fixation hooks deployed into the myocardium.

FIG. 10 illustrates a longitudinal cross-sectional view of a cardiac lead with pushably attachable fixation hooks according to an embodiment of the present invention.

FIG. 11 illustrates a cross-sectional view of FIG. 10.

FIG. 12 illustrates the view of FIG. 10 with the pushably attachable active fixation hooks deployed into the myocardium.

FIG. 13 illustrates a cross-sectional view of FIG. 12.

### DETAILED DESCRIPTION OF THE DRAWINGS

References to “one embodiment”, “an embodiment”, “in embodiments”, or the like mean that the feature being referred to is included in at least one embodiment of the invention. Moreover, separate references to “one embodiment”, “an embodiment”, or “in embodiments” do not necessarily refer to the same embodiment; however, neither are such embodiments mutually exclusive, unless so stated. Thus, the invention can include any variety of combinations and/or integrations of the embodiments described herein.

As used herein “substantially”, “relatively”, “generally”, “about”, and “approximately” are relative modifiers intended to indicate permissible variation from the characteristic so modified. They are not intended to be limited to the absolute value or characteristic which it modifies but rather approaching or approximating such a physical or functional characteristic.

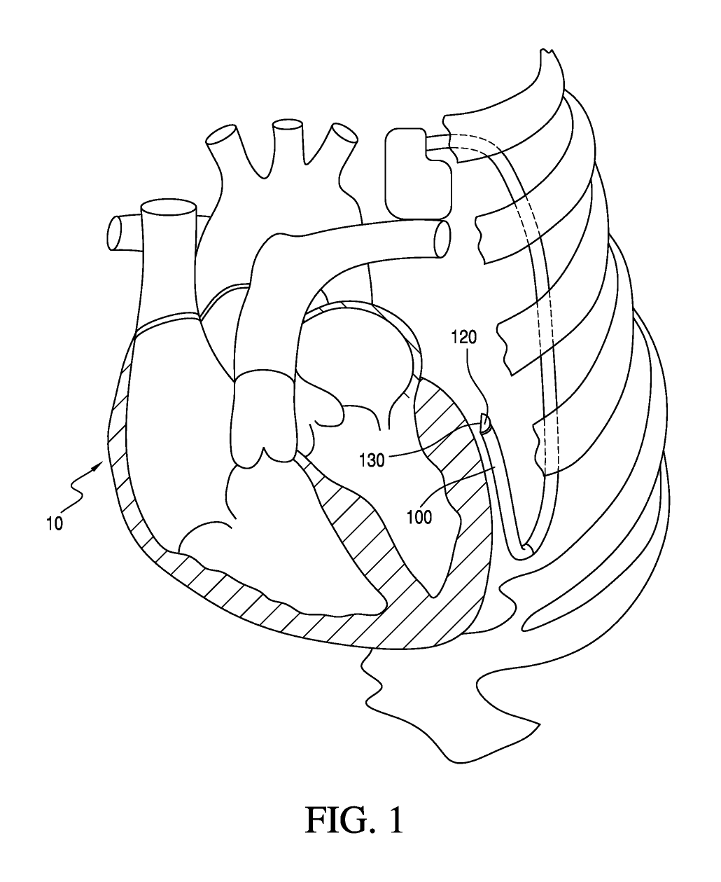

As shown in FIGS. 1-13, an implantable cardiac electrical lead according to the present invention is provided. The cardiac lead comprises an active fixation mechanism and a half-domed or semi-spherical distal tip that provides lead directionality and increased precision of lead placement. An anode and cathode may be separated at a certain distance.

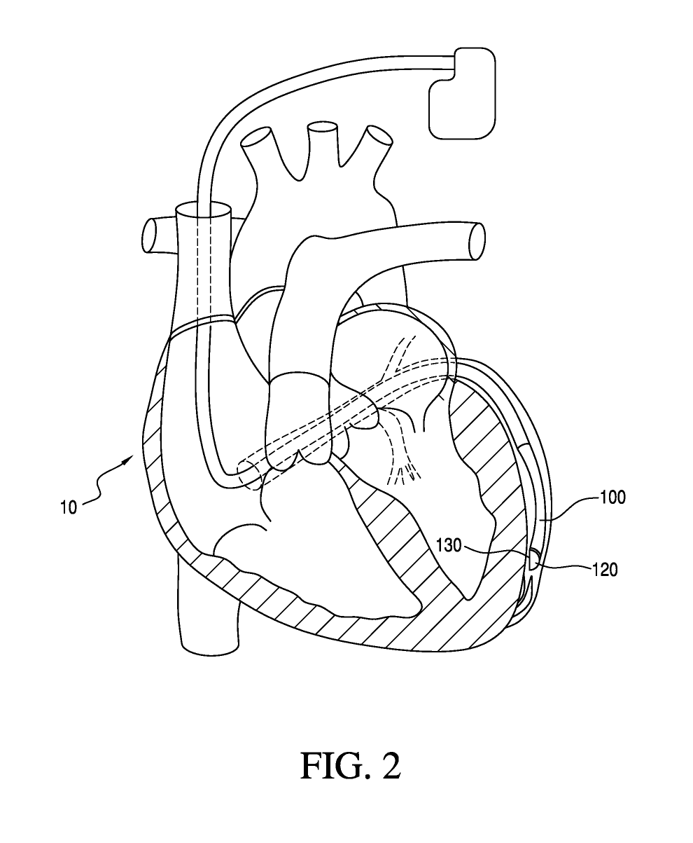

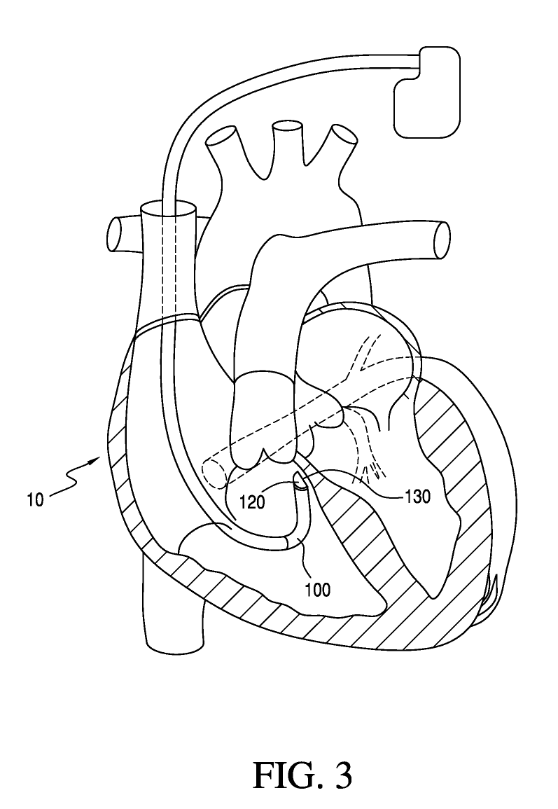

The half-domed or semi-spherical distal tip or end allows the cardiac lead to be safely and precisely placed and anchored into the myocardium in a variety of locations within and on the surface of the heart. For example, in specific embodiments, the cardiac lead may be placed on the epicardium delivered through a sheath by pericardial puncture or mini-thoracotomy (as shown in FIG. 1); inside a coronary vein intravascularly (as shown in FIG. 2); or on the ventricular septum from the right ventricle intravascularly, as delivered through a sheath by venotomy or venipuncture (as shown in FIG. 3).

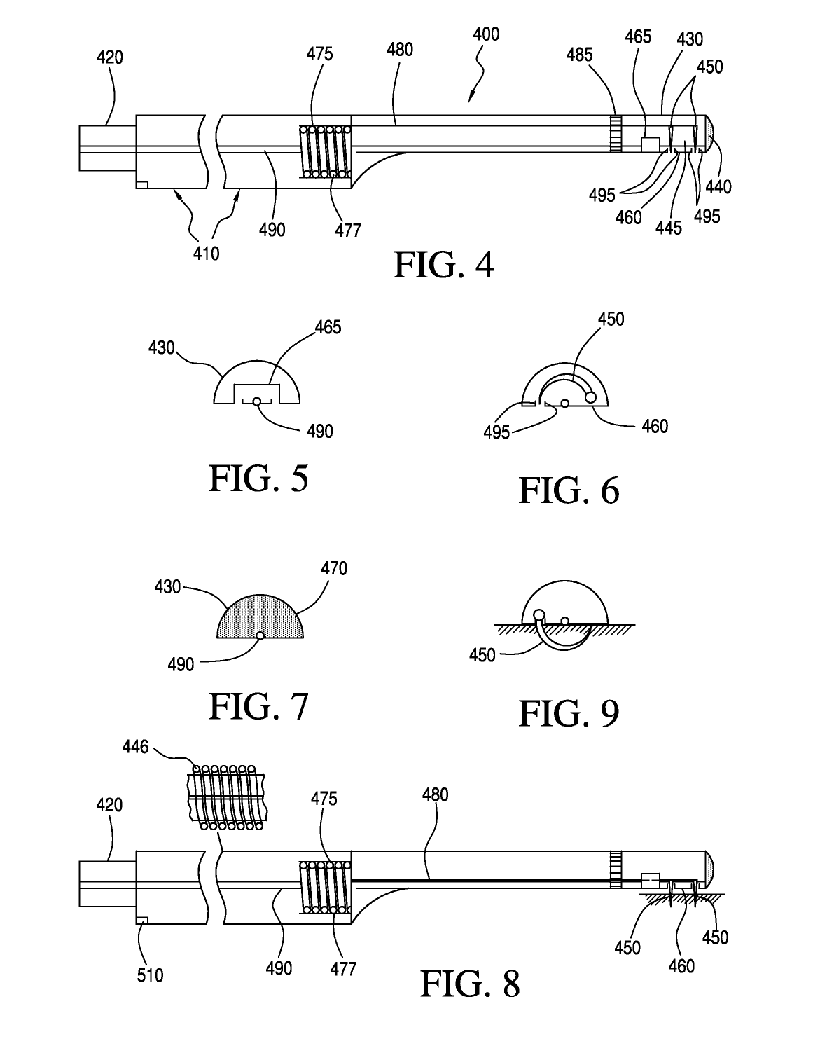

FIG. 1 is a perspective view of a cardiac lead 100 according to the present invention when implanted directly to the epicardium of heart 10. The cardiac lead 100 may be implanted through a safety sheath for example, by pericardial puncture; mini-thoracotomy by thorascope; or direct incision. Cardiac lead 100 is placed with a flat side 130 of a distal assembly having a half-domed or semi-spherical tip 120 facing the myocardium tissue. The flat side 130 of the distal assembly with the half-domed or semi-spherical tip 120 allows the operator to accurately position the active fixation mechanism (discussed below in greater detail and as shown in FIGS. 4, 8, 10, 12) towards the myocardium prior to deployment. In specific embodiments, the active fixation mechanism may comprise fixation hooks disposed within the half-domed or semi-spherical tip 120.

Due to the placement of the active fixation mechanism towards the myocardium, the active fixation mechanism can be reliably deployed into the myocardium. Further, due to the configuration of the active fixation mechanism being completely disposed within the half-domed or semi-spherical tip 120 prior to engagement, the cardiac lead 100 avoids causing any untoward injury to the adjacent tissue, especially the vessel wall. The fixation mechanism is not exposed until the operator is assured that the flat side 130 of the half-domed tip 120 is clearly positioned towards the myocardium.

In embodiments, pericardial puncture or mini-thoracotomy may be performed under fluoroscope guidance. The safety sheath may be placed first into the pericardial space. The cardiac lead 100 may be inserted into the pericardial space through the safety sheath. The cardiac lead 100 is then positioned at the intended site, for example, the site with the latest contraction. The active fixation mechanism may then be deployed with the flat side 130 facing the myocardium. During the implant, the sites with cardiac vessels coursing through the epicardium can be avoided as these sites may have very high pacing thresholds and low sensed electrical signal from the myocardium.

FIG. 2 illustrates an embodiment of a cardiac lead 100 implanted through a cardiac coronary vein. The cardiac lead 100 extends intravenously through the superior vena cava into the right atrium and then through the coronary ostium and sinus into a cardiac coronary vein. A safety sheath may be used to introduce the cardiac lead 100 into the heart. Once the flat side 130 of the cardiac lead 100 is determined to be facing the myocardium, the active fixation mechanism may be deployed.

FIG. 3 illustrates an embodiment of a cardiac lead 100 implanted into the ventricular septum from the right ventricle. The cardiac lead 100 extends through the superior vena cava into the right atrium and then through the tricuspid valve into the right ventricle. A pre-formed J-stylet with different degrees may be used to place the cardiac lead 100 onto the ventricular septum from the right ventricle. With the active fixation mechanism deployed tangentially from the flat side 130, the fixation mechanism can be accurately placed towards the myocardial tissue, for example, under a fluoroscope.

FIG. 4 illustrates a cardiac lead 400 according to at least one embodiment of the present invention. Cardiac lead 400 comprises a cylindrical body 410 having a proximal assembly 420 and a distal assembly 430.

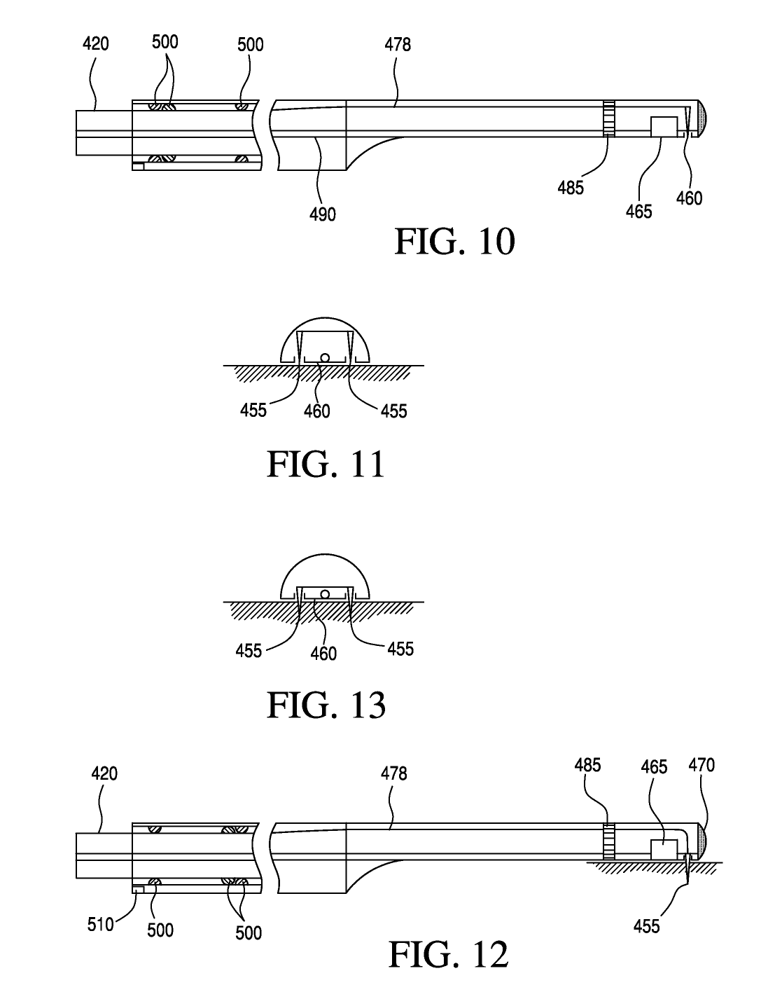

The distal assembly 430 comprises an end or tip 440 having a half-domed or semi-spherical geometry, and an active fixation mechanism 445. The active fixation mechanism is located within the distal assembly and may be deployed and retracted by an operator. In specific embodiments, the active fixation mechanism 445 may comprise rotatably attachable or fixable hooks 450 (as shown in FIGS. 6 and 8-9) or pushably attachable or fixable hooks 455 (as shown in FIGS. 10-13). The fixation mechanism is deployed from a flat side 460 of the distal assembly 430 tangentially to provide better and larger contact with the myocardium (FIGS. 6-9).

The half-domed or semi-spherical shaped end or tip 440 provides a clear directionality as to whether the fixation-mechanism-deploying flat side 460 is facing the myocardium without exposing the fixation mechanism 445 during lead placement. During the implant process, the fixation mechanism 445 is retracted inside the distal assembly 430. Thus, laceration of the myocardial or vascular structure leading to potentially significant bleeding is avoided. The fixation mechanism should not be deployed until an operator is certain that the flat side 460 of the distal assembly 430 is facing the myocardium under the fluoroscope or other imaging measures.

In embodiments, the distal assembly 430 may comprise a steroid reservoir 465 (FIGS. 4-5). The stored steroid may be released gradually after implant to prevent excessive scar formation around the active fixation mechanism. The half-domed or semi-spherical shaped tip or end 440 may comprise a coating 470 (FIGS. 4 and 7) to prevent perforation.

In specific embodiments, the cardiac lead 400 further comprises a rotatable shuttle 475, a rotatable lever/wire 480, a proximal pole 485, and a guide-wire channel 490 (FIGS. 4-5). As the cardiac lead may be placed inside a coronary vein, a hollow guide-wire channel 490, through the entire length of the cardiac lead, may be located in the center of the lead to allow a guide-wire to pass through (FIGS. 4, 7, 8, 10, and 12). For placements directly into the epicardium, or onto the ventricular septum, through a safety sheath, the hollow guide-wire channel 490 may terminate at the end of the distal assembly 430.

In embodiments, the proximal assembly 420 of the cardiac lead 400 may be designed according to current industrial standard IS-1 connector, but having a rotatable or pushable configuration.

In specific embodiments in which the active fixation mechanism comprises rotatably-attachable or fixable hooks 450, the hooks may be mounted to one side of the distal assembly 430 and attached to a rotatable lever or wire 480, as shown in FIGS. 4 and 8. The rotatable lever or wire 480 may be attached to a rotatable shuttle 475 which then is attached to a conductor 477 that is controlled by the IS-1 connector at the proximal assembly 420 (FIGS. 4 and 8).

In specific embodiments in which the active fixation mechanism comprises pushably-attachable or fixable hooks 455, the hooks may be mounted to the distal assembly 430 with one of the hooks on each side, which are then connected to a pushing lever or wire 478, as shown in FIGS. 10 and 12, which then is controlled by the IS-1 connector at the proximal assembly 420.

In specific embodiments, the half-domed shape end of the distal assembly may be coated, for example with a silicon or polyurethane coating 470, to avoid perforation of the myocardium during the process of lead placement (FIG. 7).

In specific embodiments, the active fixation mechanism 445 may be connected to or applied to a defibrillator lead. In other embodiments, a defibrillator lead 446 may be located at a point between the proximal assembly and distal assembly (FIG. 8).

Once the cardiac lead is in a desired position with the flat side of the distal end facing the myocardium in the pericardial space, coronary vein, ventricular septum or the distal apex, the active fixation mechanism can be deployed (e.g., by a rotatable-attach fixation mechanism or a pushable-attach fixation mechanism) (FIGS. 8-9 and 12-13).

In embodiments, this deployment may be accomplished by rotating the proximal assembly 420 thereby causing rotation of the rotatable shuttle 475, and thus rotation of the rotation lever or wire 480 and the hooks 450 (FIGS. 4, 6 and 8-9). Finally, the hooks are fixated into the myocardium tangentially from the flat side 460. The deployment of the hooks may be accomplished by clockwise or counterclockwise rotation and retraction may be done by reversed motion.

Alternatively, in other embodiments, the deployment may be accomplished by pushing the proximal end 420 thereby causing a forward and inward movement of the pre-formed hooks 455 from the flat side 460 of the distal assembly (FIGS. 10-13). The push-attach fixation mechanism may be preferred for placement of the cardiac lead into a coronary vein intravascularly as the vein may be small. Therefore, a stiff structure such as the rotatable shuttle and lever/wire may prove to be difficult for such placement. For the pushable-attach fixation mechanism, the proximal end 420 of the cardiac lead may comprise inner stoppers/seals 500 hermetically sealed to prevent over-pushing of the fixation hooks 455 into the myocardium and over-pulling when the hooks 455 are retracted (FIGS. 10 and 12).

In specific embodiments, the fixation hooks may be used as active distal electrodes, for example a pacing/sensing electrode, thereby providing electrical stimuli and sensing the electrical signal from the myocardium. The diameter and the length of the hooks may be determined based on the need of a surface area of about 8 mm², with or without insulation coating.

A proximal electrode may be located further away from the end of the distal assembly. A distal electrode or cathode, i.e., the active fixation hooks and the proximal pole, or the anode, may be separated to certain distance to provide adequate sensing without causing over- or under-sensing.

To minimize the effects of scarring that occur after a lead is fixated into myocardium, a steroid reservoir 465 (FIGS. 4, 5, 9 10, and 12) is assembled between the proximal electrode (anode) and the distal electrode (cathode) to release steroid to suppress local inflammation, therefore scarring.

In embodiments, a color marker 510 may be placed on the proximal assembly, corresponding with the location of the flat side of the distal assembly (FIGS. 4, 8, 10, and 12).

Although specific embodiments of the invention have been described herein, it is understood by those skilled in the art that many other modifications and embodiments of the invention will come to mind to which the invention pertains, having benefit of the teaching presented in the foregoing description and associated drawings.

It is therefore understood that the invention is not limited to the specific embodiments disclosed herein, and that many modifications and other embodiments of the invention are intended to be included within the scope of the invention. Moreover, although specific terms are employed herein, they are used only in generic and descriptive sense, and not for the purposes of limiting the description invention.

1. A cardiac lead, comprising:
a cylindrical body having a proximal assembly and a distal assembly, said distal assembly comprising:
a distal end having a half-domed or semi-spherical shape;
an active fixation mechanism housed in the distal assembly and that is deployable and retractable from a flat surface of the distal assembly; and
a steroid reservoir.

2. A cardiac lead according to claim 1, wherein the active fixation mechanism comprises:
at least one rotatable hook for fixation into heart tissue; and
at least one stopping ring hermetically sealed to prevent over-deployment of the at least one rotatable hook.

3. A cardiac lead according to claim 1, wherein the active fixation mechanism comprises a plurality of hooks.

4. A cardiac lead according to claim 1, where the active fixation mechanism comprises:
at least one pushable hook for fixation into heart tissue; and
at least one stopper or seal hermetically sealed to prevent over-deployment or retraction of the at least one pushable hook.

5. A cardiac lead according to claim 1, where the active fixation mechanism comprises a plurality of pushable hooks.

6. A cardiac lead according to claim 1, further comprising a hollow guide-wire channel extending through an entire length of the cardiac lead.

7. A cardiac lead according to claim 1, wherein the proximal assembly comprises a rotatable shuttle.

8. A cardiac lead according to claim 2, wherein the at least one rotatable hook is attached to a rotatable lever or wire.

9. A cardiac lead according to claim 3, wherein the at least one pushable hook is attached to a pushable lever or wire.

10. A cardiac lead according to claim 1, wherein the half-dome shaped or semi-spherical end is coated with a silicon or polyurethane coating.

11. A cardiac lead according to claim 1, further comprising a defibrillator lead.

12. A cardiac lead according to claim 1, wherein said active fixation mechanism comprises two hooks and said hooks serves as pacing/sensing electrodes.

13. A method for implanting a cardiac lead, comprising:
inserting the cardiac lead of claim 1 through a flexible sheath;
placing the flat surface of the distal assembly against heart tissue; and
deploying the active fixation mechanism into the heart tissue.

14. A method according to claim 13, wherein the heart tissue comprises myocardium.

15. A method according to claim 13, comprising placing the cardiac lead at a site on the epicardium with the latest contraction.

16. A method according to claim 13, comprising inserting the cardiac lead into a coronary vein.

17. A method according to claim 13, comprising inserting the cardiac lead onto a ventricular septum.

18. A method according to claim 13, wherein the active fixation mechanism comprises hooks that provide electrical stimuli to or sense electrical signals from the heart.

19. A cardiac lead, comprising:
a cylindrical body having a proximal assembly and a distal assembly, said distal assembly comprising:
a distal end having a geometric shape to provide lead directionality to heart tissue and to provide placement of means for fixing the distal end to the heart tissue, said means for fixing being deployable and retractable from a flat surface of the distal assembly.

20. A cardiac lead according to claim 19, further comprising means for suppressing inflammation as the cardiac lead is fixated to the heart tissue.
